(19) United States
(12) Patent Application Publication
(10) Pub. No.: US 2010/0262117 A1
(43) Pub. Date: Oct. 14, 2010

(54) PREDICTIVE CONTROL BASED SYSTEM AND METHOD FOR CONTROL OF INSULIN DELIVERY IN DIABETES USING GLUCOSE SENSING

(75) Inventors: Lalo Magni, Vellezzo Bellini (IT); Giuseppe De Nicolao, Milan (IT); Davide Martino Raimondo, Albuzzano (IT); Claudio Cobelli, Padova (IT); Chiara Dalla Man, Venezia (IT)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/740,275

(22) PCT Filed: Oct. 31, 2008
(86) PCT No.: PCT/US2008/082063

§ 371 Date: Apr. 28, 2010

Related U.S. Application Data
(60) Provisional application No. 60/984,956, filed on Nov. 2, 2007.

(51) Int. Cl. A61M 37/00

(52) U.S. Cl. 604/504; 604/66

(57) ABSTRACT

A system and method for providing optimal insulin injections to a subject, using a controller, a continuous glucose monitor, and an insulin delivery unit is disclosed. The controller possesses a discrete-time, linear model predictive control law, means for sending information to the insulin delivery unit, and means for receiving information from the CGM. The control law implemented is derived from a discrete-time model of glucose insulin dynamics and an aggressiveness parameter. The result is that using only glucose measurements obtained from sensor readings and, prior values of external insulin infusion and meal and exercise announcement the optimal insulin injection necessary to safely regulate blood glucose can be calculated.

### RELATED APPLICATIONS

The present invention claims priority from U.S. Provisional Application Ser. No. 60/984,956, filed Nov. 2, 2007, entitled “Model Predictive Control Based Method for Closed-Loop Control of Insulin Delivery in Diabetes Using Continuous Glucose Sensing” of which is hereby incorporated by reference herein in its entirety.

The present invention is related to PCT Application No. PCT/US2008/067725, filed Jun. 20, 2008, entitled “Method, System and Computer Simulation Environment for Testing of Monitoring and Control Strategies in Diabetes,” of which is hereby incorporated by reference.

### FIELD OF THE INVENTION

Some aspects of this invention are in the field of glycemic control. More specifically, the invention provides a novel method and system to compute an optimal adapting insulin injection based on continuous glucose monitoring. More particularly, the invention or aspects thereof use glucose measures obtained in the previous glucose samples, the previous values of the external insulin infusion, and meal and exercise announcements to compute the optimal insulin injection to safely regulate glucose concentration.

### BACKGROUND OF THE INVENTION

### Importance of Glycemic Control in Diabetes

In health, blood glucose (BG) is tightly controlled by a hormonal network that includes the gut, liver, pancreas and brain, ensuring stable fasting BG levels (˜80-100 mg/dl) and transient postprandial glucose fluctuations. Diabetes is a combination of disorders characterized by absent or impaired insulin action, resulting in hyperglycemia. Intensive insulin and oral medication treatment to maintain nearly normal levels of glycemia markedly reduces chronic complications in both Type 1 (T1DM, [dcctrg93]) and Type 2 diabetes (T2DM, [ukpds98]), but may cause a risk of potentially life-threatening severe hypoglycemia (SH). This SH results from imperfect insulin replacement, which may reduce warning symptoms and hormonal defenses [gold93]. Consequently hypoglycemia has been identified as the primary barrier to optimal diabetes management [cryer02].

### Early Control Strategies

Glucose control has been studied for more than 3 decades now and widely different solutions have been proposed. It is only very recently that technology and algorithm have come together to enable glucose control outside of the ICU of a hospital. The earliest work was based on intravenous (IV) glucose measure and both positive (glucose) and negative (insulin) control actuation. Studies by Pfeiffer and Clemens created systems like the GCIIS [1] or the more well known Biostator [2] that have since been used in hospital settings. Both of these regulators were based on a proportional integral derivative strategy (PID); the injected insulin is proportional to the difference between a fixed plasma glucose target and the measured plasma glucose as well as to the rate of change of plasma glucose. A different type of controller was also designed at that time, based instead on prediction of glucose, therefore counteracting the inherent inertia of exogenous insulin compared to the endogenous hormones. The major designs can be found in [3,4,5,6,7]. More work followed these initial successes, spanning a broader range of control theory. All were concerned with IV sensing and IV action, and most of them relied on some approximate modeling of human physiology. Techniques like pole placement [8], adaptive control [9], time-domain [10], worst case frequency domain (H ∞) [15], and optimization of linear quadratic costs (LQ) [11,12,13,14], were adapted to the particular case of glucose control.

### Self Monitoring of Blood Glucose (SMBG)-Based Diabetes Management

The current management of diabetes typically uses SMBG to adjust the dosing of insulin delivered via injections or insulin pump. Glucose is measured at infrequent (less than five times per day) and irregular times during the day and insulin is injected subcutaneously according to both these measures and the estimated amount of carbohydrates ingested. Depending on the treatment strategy the insulin is either injected continuously (basal rate) on discretely (boluses) via a pump, or only discretely, via injections containing both fast acting and long acting insulin. In both cases relation between the amount of insulin injected and the measured plasma glucose is determined by the care practitioner and the patient based on past experience and initial rule of thumbs (1800-rule and 450-rule). Insulin boluses are traditionally calculated in two phases: first, the amount of insulin is computed that is needed by a person to compensate for the carbohydrate content of an incoming meal. This is done by estimating the amount of carbohydrates to be ingested and multiplying by each person's insulin/carbohydrate ratio. Second, the distance between actual blood glucose (BG) concentration and individual target level is calculated and the amount of insulin to reach the target is computed. This is done by multiplying the (BG-target) difference by individual insulin correction factor. It is therefore evident that a good assessment of each person's carbohydrate ratio and correction factor is critical for the optimal control of diabetes.

### The Subcutaneous-Subcutaneous (SC-SC) Route

Since the advent of new technologies in glucose sensing and insulin infusion it is now possible to observe and act upon the glucose/insulin levels using real-time measurements, the sampling frequency of most meters being smaller or equal to 5 minutes. Therefore, increasing scientific and industrial effort are focused on the development of regulation systems (e.g. artificial pancreas) to control insulin delivery in people with diabetes.

While these new technologies do open the way to both open and closed loop control of plasma glucose, they also suffer from serious drawbacks: First, the continuous sensors currently available experience delays estimated between 10 and 20 minutes. Additionally, the continuous sensors' accuracy is still lower than, for example, finger stick measurement (SMBG) and therefore none of the currently available sensors have been approved for ‘replacement’ by the Food & Drugs Administration (FDA). This precludes their use as such in clinical decisions. Finally, subcutaneous injection of insulin imposes an additional actuation delay, the exogenous insulin being first transported from the injection site to the central vascular system and only then following the pathway of exogenous IV injected insulin.

Most recent control efforts have been focusing on the SC-SC route as it is the most likely to be easily mass marketed and it relies on readily available technologies.

### Implantable Devices

In the last decades advances in implantable sensors and insulin pumps have triggered great interest in the glucose control community [20,21,22]. The implantable sensor (directly into and artery) is believed to be closer to the classic IV sensing, and is therefore less inclined to exhibit delays and errors. Recent studies have shown that even though these sensors directly sample blood they nevertheless suffer from delays equivalent to (if a little shorter than) SC sensors [23]. Implantable pumps are also believed to be more efficient than SC pumps, in that they more closely mimic the natural route of insulin (peritoneal injections). Contrary to external pumps, this technology has been shown to suffer from insulin aggregation [23]. Both technologies, however, suffer from difficulty of insertion (surgery is required) and limited lifetime (from 3 to 18 months) [22].

### Recent Control Efforts

Recent efforts in regulating glucose homeostasis have explored three major routes. First, results on the IV-SC route have been published by Hovorka et al. and Damiano et al., focusing on subcutaneous insulin injection but accessing glucose concentration via IV measurements. Both utilize model-predictive control (MPC) methodologies. Hovorka's group focused on a strictly negative actuation (insulin only) [19]; while Damiano's group has been developing a double actuation scheme (insulin+glucagon) [18]. Second, Pr Renard from the University of Montpellier has been developing a glucose control scheme based on implanted sensor and pump (Ip-Ip route). Finally the group led by G. Steil has been developing, in collaboration with Medtronic, a fully SC-SC based glucose regulator [27], based on the PID methodology: PD+a term proportional to the integral error (sum of past errors).

### MPC Methodology

An explicit model can be incorporated or “built in” to the controller via model predictive control (MPC). The controller compares the model predicted output with the actual output, updates the model, and calculates the next manipulated input value; the basic idea is shown in detail in FIG. 5. At each time step tₖ the previous history of glucose measurements (y) and insulin delivery rates (u) are known. An optimization problem is solved, where a set of M current and future insulin delivery rates are chosen such that the model predicted glucose values reach a desired setpoint, over a future horizon of P time steps. The insulin delivery rates are constrained between minimum and maximum values. The first insulin infusion (out of M steps) is then implemented. At the next time step tₖ₊₁ a new glucose value yₖ₊₁ is measured, the model is possibly updated to learn from discrepancies between actual and predicted values, and the optimization is repeated. How to best update the model to correct for model mismatch is one of the major challenges to MPC.

Parker et al.[17] were the first to publish an MPC approach for the management of glucose levels in type 1 diabetic patients. Their research was a simulation study that employed the Sorensenb[16] model as the “virtual patient”. They explored several approaches to model development, including: (i) direct identification from patient data, (ii) reduced order numerical models that were derived from the original compartmental model, and (iii) linearized versions of the compartmental model coupled with a state estimator. The state estimator was used for inference of the (unmeasured) meal disturbance, providing a form of feedforward control without the need for direct knowledge of the meal. They also explored the estimation of key physiologic parameters on-line, using a Kalman filter. A significantly different approach was presented by Trajanoski and Wach [37]. Their model was nonlinear and strictly empirical. In simulation studies, they identified a patient from 500 data points, sampled every two minutes. Their simulation studies considered a variety of patient conditions, and focused on 15 g and 75 g oral glucose tolerance tests. The paper by Kan et al. [38] employed a linear MPC approach and experimental data for dogs. They utilized two pumps: one delivering intravenous insulin and the other intravenous glucose. Their experiments started from an initial hyperglycemic state, followed by convergence to normal glucose levels. The controller was based on a simple (fixed) first-order-plus-delay model. In comparison with a conventional PD algorithm, they claimed superior performance, although the results were subject to interpretation.

It should be noted that MPC is a basic strategy or concept, but any number of model types can be used, with many different methods of performing the optimization. Classic MPC uses a fixed linear model, but there have been many formulations using nonlinear models [24-25-26-28-29-30-39], including artificial neural networks [40]. A nice feature of an optimization-based approach is that different weighting on the control objective can be used depending on whether the glucose is entering hyperglycemia or hypoglycemia conditions. Thus, the long-term problems associated with hyperglycemia can be traded off against the short-term risks of hypoglycemia. Also, multi-objective optimization techniques can be used to rank the important objectives; for example, the highest ranked objective might be to avoid hypoglycemia.

### BRIEF SUMMARY OF INVENTION

An aspect of various embodiments of the present invention comprises, but is not limited thereto, the following: a method and system to compute an optimal adapting insulin injection based on continuous glucose monitoring.

Using only the glucose measures obtained in previous samples, previous values of the external insulin infusion and the meal and exercise announcements it computes the optimal insulin injection to safely regulate the glucose concentration. Some advantages of this input-output MPC scheme are (but not limited thereto) that an observer is not required, and that it is easily implementable because real-time optimization is avoided. Additionally, only the weight on the glucose concentration error needs to be tuned in a quite straightforward and intuitive way. The control algorithm may be based on a population model of the meal-insulin-glucose system (see e.g. the model introduced in [31] for normal subjects and modified for diabetic patients in [42]). A tool to verify the performance of the controller is used to adapt the tuning of the controller to physiological changes.

An aspect of various embodiments of the present invention (or partial embodiments, combinations of various embodiments in whole or in part) may provide a number of novel and nonobvious features, elements and characteristics, such as but not limited thereto closed-loop control of insulin delivery based on continuous glucose sensing with the following characteristics: a population model is used; only a unique model with the mean value of the parameters is used for the synthesis of the regulator; meal announcement is used in advance; on-line optimization is avoided; an auto-tuning tool is incorporated for adapting the tuning of the controller; the auto-tuning tool is based on suitable patient's feature and a function derived from the virtual patients obtained from the population model; the features are either clinical parameters or parameters obtained from insulin and glucose data collected during a screening visit; and sampling time can be changed during the day.

An aspect of an embodiment of the present invention (or partial embodiment, combinations of various embodiments in whole or in part) comprises a system for providing optimal insulin injections to a subject to be used with a continuous glucose monitor (CGM) and an insulin delivery unit. The system comprising: a controller. The controller may comprise: a discrete-time, linear model predictive control law, means for sending information to the insulin delivery unit, and means for receiving information from the CGM. The process and related means may be implemented using hardware, software of a combination thereof and may be implemented, for example, in one or more computer systems or other processing systems.

An aspect of an embodiment of the present invention (or partial embodiment, combinations of various embodiments in whole or in part) comprises a computer method for providing optimal insulin injections to a subject to be used with a continuous glucose monitor (CGM) and an insulin delivery unit. The method comprising: providing a discrete time linear model predictive control law, sending information to an insulin delivery unit, and receiving information from the CGM.

An aspect of an embodiment of the present invention (or partial embodiment, combinations of various embodiments in whole or in part) comprises a computer readable medium for use with a processor, to be used with a continuous glucose monitor (CGM) and an insulin delivery unit. The processor having computer executable instructions for performing a method for computing an optimal adapting insulin injection. The method comprising: providing a discrete time linear model predictive control law, sending information to an insulin delivery unit, and receiving information from the CGM.

An aspect of an embodiment of the present invention (or partial embodiment, combinations of various embodiments in whole or in part) comprises a system and method for providing optimal insulin injections to a subject, using a controller, a continuous glucose monitor, and an insulin delivery unit is disclosed. The controller possesses a discrete-time, linear model predictive control law, means for sending information to the insulin delivery unit, and means for receiving information from the CGM. The control law implemented is derived from a discrete-time model of glucose insulin dynamics and an aggressiveness parameter. The result is that using only glucose measurements obtained from sensor readings and, prior values of external insulin infusion and meal and exercise announcement the optimal insulin injection necessary to safely regulate blood glucose can be calculated.

These and other objects, along with advantages and features of the invention disclosed herein, will be made more apparent from the description, drawings and claims that follow.

### BRIEF DESCRIPTION OF THE DRAWINGS

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 illustrates a block diagram of a glucose management system for practicing one or more embodiments of the present invention using unidirectional wired connections for communications.

FIG. 2 illustrates a block diagram of a glucose management system for practicing one or more embodiments of the present invention using unidirectional wireless connections for communications.

FIG. 3 illustrates a block diagram of a glucose management system for practicing one or more embodiments of the present invention using bidirectional wired connections for communications.

FIG. 4 illustrates a block diagram of a glucose management system for practicing one or more embodiments of the present invention using bidirectional wireless connections for communications.

FIG. 5 illustrates the workings of a system that implements model predictive control.

FIG. 6 illustrates a system in which one or more embodiments of the invention can be implemented using a network, or portions of a network or computers.

FIG. 7 illustrates an exemplary computing device having computer-readable instructions in which one or more embodiments of the invention can be implemented.

FIG. 8 illustrates a block diagram of a glucose management system for practicing one or more embodiments of the present invention wherein a continuous glucose monitor and controller are physically connected.

FIG. 9 illustrates a block diagram of a glucose management system for practicing one or more embodiments of the present invention wherein a controller and insulin pump are physically connected.

FIG. 10 illustrates a block diagram of a glucose management system for practicing one or more embodiments of the present invention wherein a continuous glucose monitor, controller, and insulin pump are physically connected.

FIG. 11 illustrates a block diagram of the derivation of a model predictive control law as used in one or more embodiments of the present invention.

### DETAILED DESCRIPTION OF THE INVENTION

As described in further detail below, in accordance with the various embodiments of the present invention, there is provided a method, system and computer program product for delivering optimal insulin injections to a subject. In particular, within the scope of the present invention, there are provided methods and systems for the use of a continuous glucose monitor, and insulin delivery unit, and a controller that provide optimum insulin injections. Methods providing for a computer program product for determining an optimal insulin injection are also disclosed.

It should be appreciated that any of the components or sub-components discussed herein with regards to the various embodiments of the present invention may be communicated with one another with data or signal transfer via a variety of communications interfaces. For instance, in the form of signals or data may be electronic, electromagnetic, optical or other signals capable of being received by communications interface and components and subcomponents of the present invention. For instance, the communications may be implemented using wire or cable, fiber optics, a phone line, a cellular phone link, an RF link, an infrared link, and other communications channels (hard wire or wireless).

Similarly, any material, fluid or medium transported between components or sub-components discussed herein with regards to the various embodiments of the present invention may include a variety of types, such as, but not limited thereto, the following: conduits, tubes, lumens, channels, needles, catheters or the like.

Some illustrative and non-limiting components of the system and related method includes controller, insulin deliver device/unit, glucose monitor (e.g., CGM or SMBG), pump, computer, processor, memory, user interface(s)—local or remote or combination—, networks, printer, recorder, compiler, etc.

Any of the components or sub-components may also be controlled by voice activation.

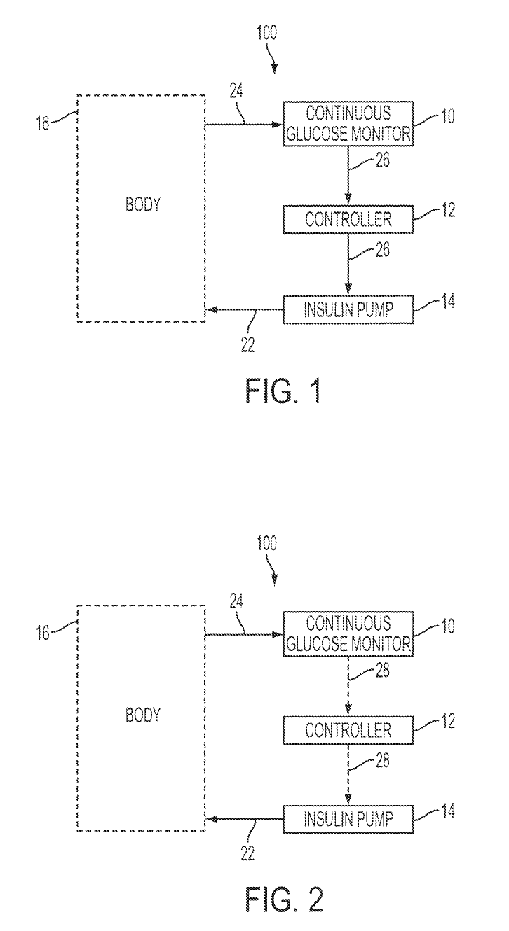

FIG. 1 illustrates a system 100 for delivering optimal insulin injections in accordance with one or more embodiments of the present invention. Continuous glucose monitor (CGM) 10 takes a reading from body 16 that includes information in the form of glucose level 24. The body may be, for example, a human subject. CGM 10 may be any continuous glucose monitor/sensor such as the Navigator from Abbott Diabetes Care, the Dexcom from Dexcom, Inc., or the Guardian/Paradigm from Medtronic, or any other commercially available continuous glucose monitor/sensor. CGM 10 then communicates to controller 12 through a unidirectional wired connection 26. Unidirectional wired connection 26 may take the form of coaxial cable, fiber optic cable, or any other means of wired communications. Controller 12 communicates with insulin pump 14 through another unidirectional wired connection 26, leading insulin pump 14 to deliver insulin 22 to the body 16. The insulin pump may be any insulin pump, including those commercially available such as the Omnipod from Insulet or the Deltec Cozmo from Smiths Medical, as well as any other insulin delivering unit.

FIG. 2 illustrates a system 100 for delivering optimal insulin injections in accordance with one or more embodiments of the present invention. CGM 10 takes a reading from body 16 that includes information in the form of glucose level 24. The body may be, for example, a human subject. CGM 10 may be any continuous glucose monitor/sensor such as the Navigator from Abbott Diabetes Care, the Dexcom from Dexcom, Inc., or the Guardian/Paradigm from Medtronic, or any other commercially available continuous glucose monitor/sensor. CGM 10 then communicates to controller 12 through a unidirectional wireless connection 28. Unidirectional wireless connection 28 may take the form of 802.11x, Bluetooth, RF, or any means of wireless communications. Controller 12 communicates with insulin pump 14 through another unidirectional wireless connection 28, leading insulin pump 14 to deliver insulin 22 to the body 16. The insulin pump may be any insulin pump, including those commercially available such as the Omnipod from Insulet or the Deltec Cozmo from Smiths Medical, as well as any other insulin delivering unit.

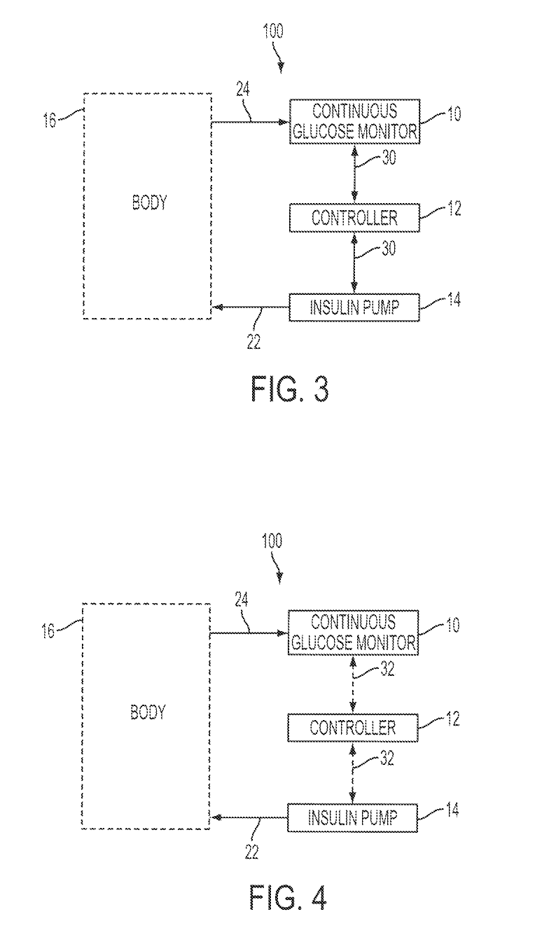

FIG. 3 illustrates a system 100 for delivering optimal insulin injections in accordance with one or more embodiments of the present invention. CGM 10 takes a reading from body 16 that includes information in the form of glucose level 24. The body may be, for example, a human subject. CGM 10 may be any continuous glucose monitor/sensor such as the Navigator from Abbott Diabetes Care, the Dexcom from Dexcom, Inc., or the Guardian/Paradigm from Medtronic, or any other commercially available continuous glucose monitor/sensor. CGM 10 then communicates to controller 12 through a bidirectional wired connection 30. Bidirectional wired connection 30 may take the form of coaxial cable, fiber optic cable, or any other means of wired communications. Controller 12 communicates with insulin pump 14 through another bidirectional wired connection 30, leading insulin pump 14 to deliver insulin 22 to the body 16. The insulin pump may be any insulin pump, including those commercially available such as the Omnipod from Insulet or the Deltec Cozmo from Smiths Medical, as well as any other insulin delivering unit.

FIG. 4 illustrates a system 100 for delivering optimal insulin injections in accordance with one or more embodiments of the present invention. CGM 10 takes a reading from body 16 that includes information in the form of glucose level 24. The body may be, for example, a human subject. CGM 10 may be any continuous glucose monitor/sensor such as the Navigator from Abbott Diabetes Care, the Dexcom from Dexcom, Inc., or the Guardian/Paradigm from Medtronic, or any other commercially available continuous glucose monitor/sensor. CGM 10 then communicates to controller 12 through a bidirectional wireless connection 32. Bidirectional wireless connection 32 may take the form of 802.11x, Bluetooth, RF, or any means of wireless communications. Controller 12 communicates with insulin pump 14 through another bidirectional wireless connection 32, leading insulin pump 14 to deliver insulin 22 to the body 16. The insulin pump may be any insulin pump, including those commercially available such as the Omnipod from Insulet or the Deltec Cozmo from Smiths Medical, as well as any other insulin delivering unit.

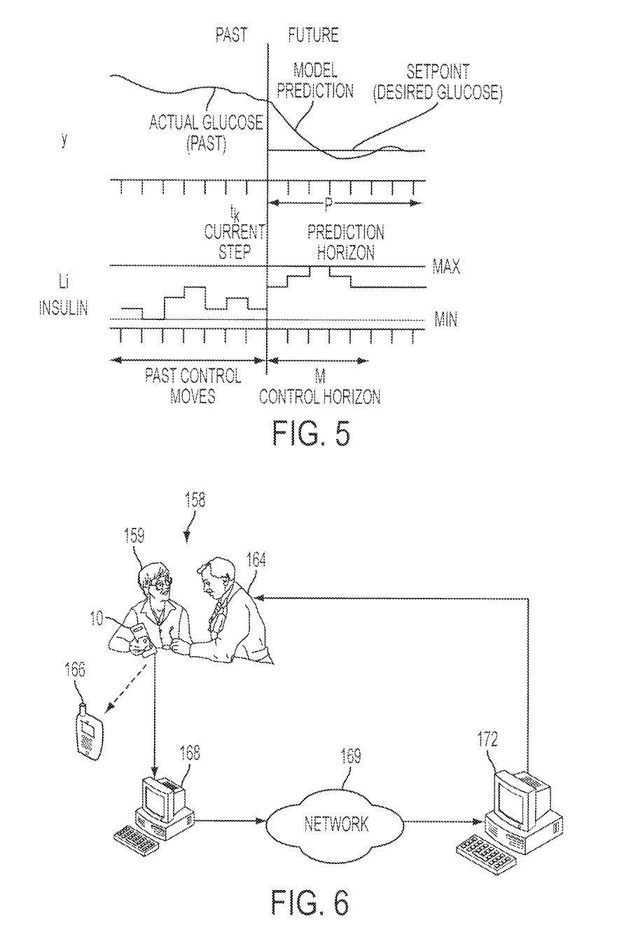

FIG. 5 illustrates the workings of a system that implements model predictive control. Such a system may be used to achieve a desired glucose level in a subject according to the present invention. The controller compares the model predicted output with the actual output, updates the model, and calculates the next manipulated input value. At each time step tₖ the previous history of glucose measurements (y) and insulin delivery rates (u) are known. An optimization problem is solved, where a set of M current and future insulin delivery rates are chosen such that the model predicted glucose values reach a desired setpoint, over a future horizon of P time steps. The insulin delivery rates are constrained between minimum and maximum values. The first insulin infusion (out of M steps) is then implemented. At the next time step tₖ₊₁ a new glucose value yₖ₊₁ is measured, the model is possibly updated to learn from discrepancies between actual and predicted values, and the optimization is repeated.

FIG. 6 diagrammatically illustrates an exemplary system in which examples of the invention can be implemented. Referring to FIG. 6, clinic setup 158 provides a place for doctors (e.g. 164) to diagnose patients (e.g. 159) with diseases related with glucose. CGM (or sensing device incorporating glucose testing function) 10 can be used to monitor and/or test the glucose levels of the patient. It should be appreciated that while only CGM 10 is shown in the figure, the system of the invention and any component thereof may be used in the manner depicted by FIG. 6. The system or component may be affixed to the patient or in communication with the patient as desired or required. For example the system or combination of components thereof—including CGM 10, controller 12, or insulin pump 14, or any other device or component—may be affixed to the patient through tape or tubing or may be in communication through wired or wireless connections. Such monitor and/or test can be short term (e.g. clinical visit) or long term (e.g. clinical stay or family). The CGM outputs can be used by the doctor for appropriate actions, such as insulin injection or food feeding for the patient, or other appropriate actions. Alternatively, the CGM output can be delivered to computer terminal 168 for instant or future analyses. The delivery can be through cable or wireless or any other suitable medium. The CGM output from the patient can also be delivered to a portable device, such as PDA 166. The CGM outputs with improved accuracy can be delivered to a glucose monitoring center 172 for processing and/or analyzing. Such delivery can be accomplished in many ways, such as network connection 170, which can be wired or wireless.

In addition to the CGM outputs, errors, parameters for accuracy improvements, and any accuracy related information can be delivered, such as to computer 168, and/or glucose monitoring center 172 for performing error analyses. This can provide a centralized accuracy monitoring and/or accuracy enhancement for glucose centers, due to the importance of the glucose sensors.

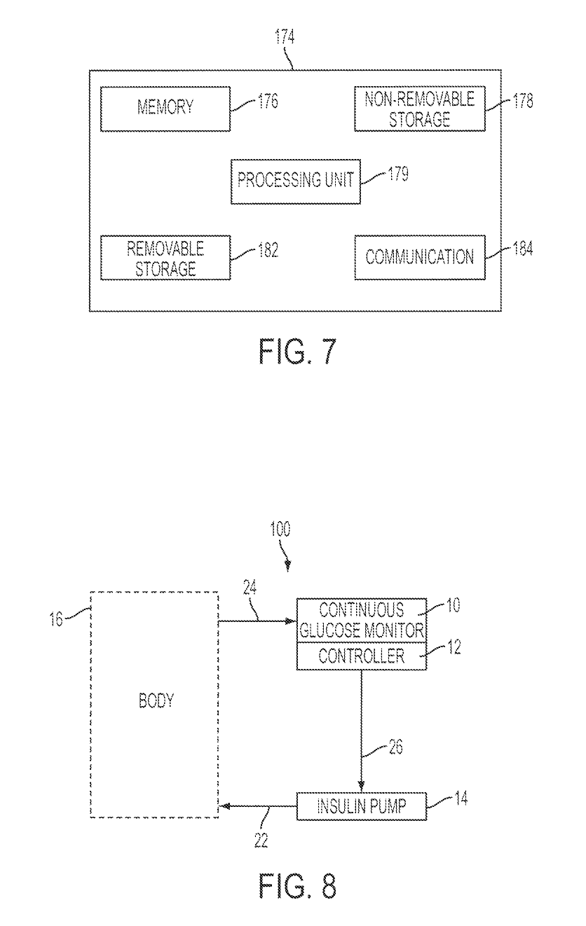

Examples of the invention can also be implemented in a standalone computing device associated with the target CGMs. An exemplary computing device in which examples of the invention can be implemented is schematically illustrated in FIG. 7. Although such devices are well known to those of skill in the art, a brief explanation will be provided herein for the convenience of other readers. Referring to FIG. 7, in its most basic configuration, computing device 174 typically includes at least one processing unit 179 and memory 176. Depending on the exact configuration and type of computing device, memory 176 can be volatile (such as RAM), non-volatile (such as ROM, flash memory, etc.) or some combination of the two.

Additionally, device 174 may also have other features and/or functionality. For example, the device could also include additional removable and/or non-removable storage including, but not limited to, magnetic or optical disks or tape, as well as writable electrical storage media. Such additional storage is represented by removable storage 182 and non-removable storage 178. Computer storage media includes volatile and nonvolatile, removable and non-removable media implemented in any method or technology for storage of information such as computer readable instructions, data structures, program modules or other data. The memory, the removable storage and the non-removable storage are all examples of computer storage media. Computer storage media includes, but is not limited to, RAM, ROM, EEPROM, flash memory or other memory technology, CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of, or used in conjunction with, the device.

The device may also contain one or more communications connections 184 that allow the device to communicate with other devices (e.g. other computing devices). The communications connections carry information in a communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. As discussed above, the term computer readable media as used herein includes both storage media and communication media.

FIG. 8 illustrates a system 100 for delivering optimal insulin injections in accordance with one or more embodiments of the present invention. The configuration of system 100 is such that CGM 10 and controller 12 are physically connected to one another. In one variant, controller 12 is embedded within the physical housing of CGM 10, in another CGM 10 is embedded within the physical housing of controller 12, and in yet another the CGM 10 and controller 12 are in separate physical housings, and the physical housings are connected.

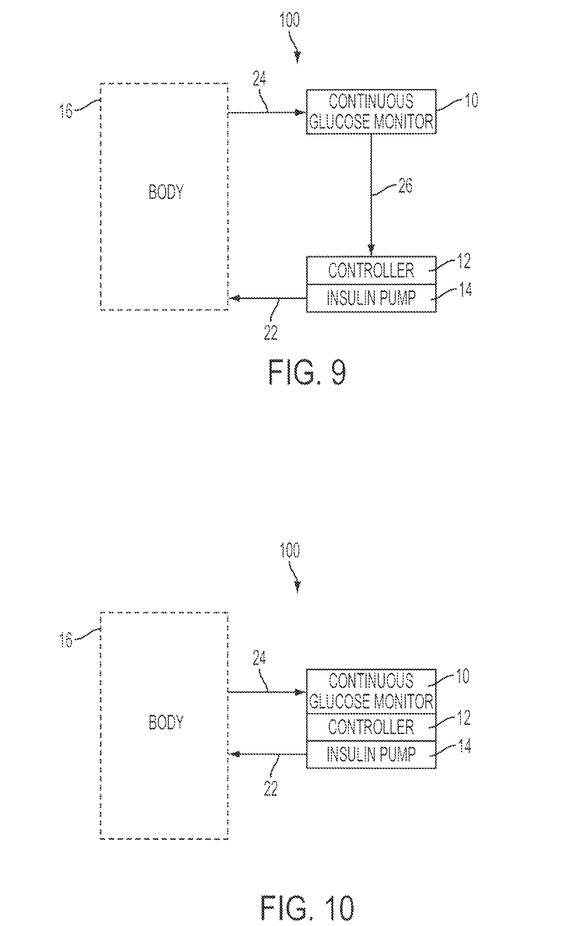

FIG. 9 illustrates a system 100 for delivering optimal insulin injections in accordance with one or more embodiments of the present invention. The configuration of system 100 is such that controller 12 and insulin pump 14 are physically connected to one another. In one variant, controller 12 is embedded within the physical housing of insulin pump 14, in another insulin pump 14 is embedded within the physical housing of controller 12, and in yet another insulin pump 14 and controller 12 are in separate physical housings, and the physical housings are connected.

FIG. 10 illustrates a system 100 for delivering optimal insulin injections in accordance with one or more embodiments of the present invention. The configuration of system 100 is such that CGM 10, controller 12, and insulin pump 14 are physically connected to one another. In one variant, CGM 10 and controller 12 are embedded within the physical housing of insulin pump 14, in another CGM 10 and insulin pump 14 are embedded within the physical housing of controller 12, in another variant insulin pump 14 and controller 12 are embedded within the physical housing of CGM 10, finally, in yet another variant, each of CGM 10, controller 12, and insulin pump 14 are in separate physical housings and the physical housings are connected.

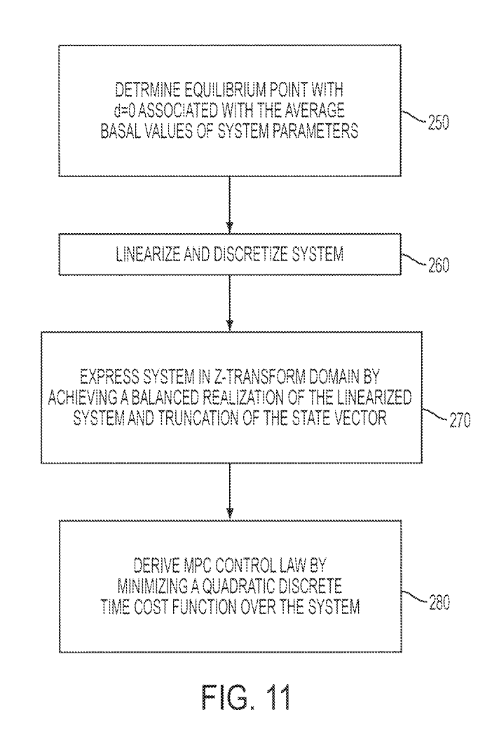

FIG. 11 diagrams the process of deriving a model predictive control law, as may be implemented in one or more embodiments of the present invention. First, the system must determine an equilibrium point with d=0 associated with the average basal values of system parameters 250. The point d=0 may represent, for example, the condition of no glucose disturbances (such as through meals). The next step is to linearize and discretize the system 260. Next, express the system in the z-transform domain by achieving a balanced realization of the linearized system and truncation of the state vector 270. This may be accomplished, for example through the use of a tool such as MATLAB, using the Control Systems Toolbox instruction modred. Finally, derive the model predictive control law by minimizing a quadratic discrete time cost function over the system 280.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

### Examples and Experimental Results

Practice of the invention will be still more fully understood from the following examples and experimental results, which are presented herein for illustration only and should not be construed as limiting the invention in any way.

### Concise Description of the Control Algorithm

Our control strategy has two main components. The first component, which entails patient assessment and individual tuning of control parameters, is done prior to a closed-loop control study using patient data collected during a screening. The second component, which entails controller warm-up and run-rime operation, includes initialization of controller state variables and run-time computation of insulin doses based on CGM measurements.

At the center of our control algorithm is a discrete-time, linear, model predictive control (MPC) law, with insulin commands taking the form of one-minute boluses (other longer or short durations may be applied as desired or required) applied every 15 minutes (other longer or short durations may be applied as desired or required). The control law is derived from:
1. A discrete-time model of glucose insulin dynamics that describes deviations from the patient's fasting glucose concentration G_{b} and basal insulin rate u_{b}. (The model itself is represented by state space equations. The equations may change upon whether the patient is a child, adolescent, or adult.)
2. An aggressiveness parameter q that is determined from patient screening data.

### Component 1—Screening

Data from screening is used in preparing the MPC control law for individualized use. In order to assess an appropriate aggressiveness parameter q for the controller, some screening questionnaire parameters are required, such as:
1) BW=θ₁: patient's body weight (kg).
2) TDI=θ₂: patient's average total daily utilization of insulin (U).
3) CF=MD=θ₃: patient's correction factor, computed as the drop in blood glucose concentration due to one unit of insulin (mg/U).
4) CR=θ₄: patient's carbohydrate ratio (g/U).
5) AUC(G)=θ₅: area under plasma glucose curve, measured during a given test (MGTT, OGTT) (mg/dl.min).
6) AUC(G-Gₚᵣₑ)=θ₆: area under plasma glucose curve above the pre-test glucose concentration, measured during a given test (MGTT, OGTT) (mg/dl.min).
7) AUC(I)=θ₇: area under plasma insulin curve, measured during a given test (MGTT, OGTT) (pmol/l.min).
8) AUC(I-Iₚᵣₑ)=θ₈: area under plasma insulin curve above the pre-test insulin concentration, measured during a given test (MGTT, OGTT) (pmol/l.min).
9) ΔG=θ₉: difference between peak and pre-test plasma glucose concentrations, measured during a given test (MGTT, OGTT) (mg/dl).
10) ΔI=θ₁₀: difference between peak and pre-test plasma insulin concentrations, measured during a given test (MGTT, OGTT) (pmol/l).
11) T=θ₁₁: time needed to glucose concentration to come back to the target after a given test (MGTT, OGTT) (min).
12) SI=θ₁₁: insulin sensitivity of the patients, measured using the oral minimal model, or similar modeling techniques (dl/kg/min per pmol/l).

From these and other possible parameters the aggressiveness parameter q is computed as:

$$q = exp ( {k}_{0} + {\sum }_{i = 1}^{n} {k}_{i} \cdot ln ( {θ}_{i} ) ) ,$$

where the regression coefficients kᵢ through k₃ are selected from a lookup table according to whether the patient is a child, adolescent, or adult. To set an appropriate reference frame for the controller, two additional screening questionnaire parameters are required:

1) G_{b}=patient's fasting glucose concentration (mg/dl), and

2) u_{b}=used as the patient's basal rate (pmol/kg/min).

Both G_{b} and u_{b} can be time-varying. The patient's body weight (kg) is in any case necessary to obtain the insulin to be injected. It is important to emphasize that all parameter estimation occurs off-line. This estimation is automated—none of the parameters of the controller are adjusted by hand. The initialization of the algorithm is therefore completed prior to the initiation of the closed-loop control portion of the study. Once this initialization is completed, there are no further parameter changes.

### Component 2—Real-Time Closed-Loop Control:

Each discrete time period (stage) of the state space model corresponds to a period that can be for example a 15 minute sampling interval (other longer or short intervals or durations may be applied as desired or required). In the following, δG(k)=G_{med}(k)−G_{b}(k) denotes the differential glucose where G_{b}(k) is the basal glucose and G_{med} is the a filtered value of the glucose concentration obtained from the CGM usually with a faster sampling (e.g. 1 minute, or rate faster or slower as desired or required) than the one used for control. δu(k)=uₙₒₘ(k)−u_{b} (k) is the differential insulin rate where u_{b}(k) is the basal insulin. At stage k, given the state vector

x(k)=[δG(k), δG(k−1), . . . , δG(k−n), δu(k−1), . . . , δu(k−n), d(k−1), . . . , d(k−n)]^{T}, n>0,

along with the vector of target glucose concentrations for the next N stages

Y⁰(k)=[y⁰(k), y⁰(k+1), . . . , y⁰(k+N)]^{T}

and the vector of future glucose disturbances

D(k)=[d(k), d(k+1), . . . , d(k+N)]^{T}

which is inferred from the patient behavioral data β collected during the screening visit, we compute the nominal MPC insulin rate

uₙₒₘ(k)=[u_{b}(k)+κ^{MPC}(x(k), Y⁰(k), D(k))]⁺,

which is designed to minimize the quadratic penalty function of a cost function. As a linear MPC, the computation is a simple closed-form expression:

κ^{MPC}(x(k), Y⁰(k), D(k))=K_{X}·x(k)+K_{Y0}·Y⁰(k)+K_{D}·D(k),

where the gain matrices are computed (in closed-form) from fixed matrices A_{IO}, B_{IO}, M_{IO}, C_{IO} and q. We compute the effective pump rate u(k) from uₙₒₘ(k) after applying several discretization and safety filters. Note that all parameters are either fixed (such as A_{IO}, B_{IO}, M_{IO}, C_{IO}) or are patient-dependent (such as q_{c}, Kₓ, K_{Y0}, K_{D}, G_{b} and u_{b}) and computed off-line according to the fixed algorithmic processes outlined in component 1 above.

Given x(k), Y⁰(k), D(k), G_{b}(k), u_{b}(k) and BW, the nominal MPC insulin rate uₙₒₘ (k) is computed through the application of linear MPC gain matrices K_{X}, K_{Y0}, K_{D} Safety limits are applied to modify uₙₒₘ(k). These safety limits may include, for example, ensuring that (1) no more than about 10 units of bolus (other magnitudes may be applied as desired or required) insulin per hour [or other rates as desired or required] (not counting basal insulin) are applied within about 2 hours (other longer or short durations may be applied as desired or required) of a meal (2) no more than about 3 units of bolus insulin (other magnitudes may be applied as desired or required) are applied within any other about 1 hour period (other longer or short durations may be applied as desired or required) and (3) basal rate should never exceed about 150% (in instant approach, but other rates may be implemented if desired or required) of the patient specified basal rate per hour block (sliding window). The resulting “safe” pump rate is denoted u_{nom,safe}(k). Next, the actual pump command U(k) is expressed as a one-minute bolus. Since pumps (see e.g. both the Deltec Cozmo and Insulet OmniPod pumps) have a bolus finite resolution, the final value of U(k) is computed to minimize the total discretization error accumulated up to stage k of the process.

### Detailed Description of the Control Algorithm

In order to synthesize the controller, a population model of the T1DM is required see e.g. [31], [42]. It should be noted that the above model is used in PCT Application No. PCT/US2008/067725 entitled “Method, System and Computer Simulation Environment for Testing of Monitoring and Control Strategies in Diabetes” filed Jun. 20, 2008 to simulate the glucose insulin systems of proxy test subjects. The methods and systems of the present invention may be implemented with any of the aspects disclosed in PCT Application No. PCT/US2008/067725.

The glucose metabolism model can be written in the following compact way:

\dot{x}(t)=f(t,x(t), u(t), d(t))

y(t)=G(t) (10)

where x is the vector of state variables, u(pmol/Kg/min) represents administration (bolus and infusion) of insulin, d(mg/min) is the rate of ingested glucose and G (mg/dl) is the subcutaneous glucose concentration. In the following, it is assumed that meal announcement is available, i.e. the disturbance signal d (the meal) is known in advance. The MPC control law is based on the solution of a Finite Horizon Optimal Control Problem (FHOCP), where a cost function J( x̅,u) is minimized with respect to the input u subject to the state dynamics of a model of the system. Letting u⁰ be the solution of the FHOCP, according to the Receding Horizon paradigm, the feedback control law u=κ^{MPC} (X) is obtained by applying to the system only the first element of the optimal solution. This way, a closed-loop control strategy is obtained solving an open-loop optimization problem.

MPC control laws can be formulated for both discrete- and continuous-time systems. The MPC is here derived from a unique input-output linearized approximation of the full model based on the average population values of the parameters.

The associated equilibrium point with d=0 is indicated by ( x̅, ū, d̅, y̅). Around this equilibrium point, the system is linearized and discretized with sample time Tₛ, yielding

δx(k+1)=A_{D}δx(k)+B_{Du}δu(k)+B_{Dd}d(k)

δy(k)=C_{D}δx(k) (11)

where δx(k)=x(kTₛ)− x̅, δu(k)=u(kTₛ)−ū and δy(k)=y(kTₛ)− y̅. Then, through a model reduction step (e.g. derived through a balanced realization of the linearized system and a truncation of the state vector), the system is re-written in the z-transform domain with an input-output representation

$$Δ Y ( z ) = \frac{{N}_{U} ( z )}{DE ( z )} Δ U ( z ) + \frac{{N}_{U} ( z )}{DE ( z )} D ( z )$$

with

Nᵤ(z)=bₙ₋₁Z⁻¹+ . . . b₀

DE(z)=Zⁿ+aₙ₋₁Zⁿ⁻¹+aₙ₋₂Zⁿ⁻²+ . . . +a₀

N_{D}(z)=b_{D}_{_{n−1}}Zⁿ⁻¹+ . . . +b_{D}_{₀}

Equivalently in the discrete-time domain,

$$δ y ( k + 1 ) = - {a}_{n - 1} δ y ( k ) - {a}_{n - 2} δ y ( k - 1 ) - … - {a}_{0} δ y ( k - n + 1 ) + {b}_{n - 1} δ u ( k ) + … + {b}_{0} δ u ( k - n + 1 ) + {b}_{{D}_{n - 1}} d ( k ) + … + {b}_{{D}_{0}} d ( k - n + 1 )$$

Then the following (non-minimal) representation is used

$$\begin{matrix}{x}_{IO} ( k + 1 ) = {A}_{IO} {x}_{IO} ( k ) + {B}_{IO} δ u ( k ) + {M}_{IO} d ( k ) & ( 12 ) \\ δ y ( k ) = {C}_{IO} {x}_{IO} ( k ) & \\ where & \\ {x}_{IO} ( k ) = [ \begin{matrix}δ y ( k ) \\ ⋮ \\ δ y ( k - n + 1 ) \\ δ u ( k - 1 ) \\ ⋮ \\ δ u ( k - n + 1 ) \\ d ( k - 1 ) \\ ⋮ \\ d ( k - n + 1 )\end{matrix} ] & \\ and & \\ {A}_{IO} = [ \begin{matrix}- {a}_{n - 1} & … & - {a}_{0} & {b}_{n - 2} & … & {b}_{0} & {b}_{{D}_{n - 2}} & … & {b}_{{D}_{0}} \\ 1 & … & 0 & 0 & … & 0 & 0 & … & 0 \\ ⋮ & ⋱ & ⋮ & ⋮ & ⋱ & ⋮ & ⋮ & ⋱ & ⋮ \\ & & 1 & & & & & & \\ & & & 0 & & & & & \\ & & & & 1 & & & & \\ & & & & & 1 & & & \\ & & & & & & 0 & & \\ & & & & & & & 1 & 0\end{matrix} ] & \\ {B}_{IO} = [ \begin{matrix}{b}_{n - 1} \\ 0 \\ ⋮ \\ 0 \\ 1 \\ 0 \\ ⋮ \\ 0\end{matrix} ] , & \\ {M}_{IO} = [ \begin{matrix}{b}_{Dn - 1} \\ 0 \\ ⋮ \\ 0 \\ 1 \\ 0 \\ ⋮ \\ 0\end{matrix} ] & \\ {C}_{IO} = [ \begin{matrix}1 & 0 & … & 0\end{matrix} ] & \end{matrix}$$

In order to derive the MPC control law, the following quadratic discrete-time cost function is considered

$$J ( {x}_{IO} ( k ) , δ u ( \cdot ) ) = {\sum }_{i = 0}^{N - 1} ( q \begin{matrix}{( \begin{matrix}{y}^{0} ( k + i ) - \\ y ( k + i )\end{matrix} )}^{2} + \\ {r ( δ u ( k + i ) )}^{2}\end{matrix} ) + {s ( \begin{matrix}{y}^{0} ( k + N ) - \\ y ( k + N )\end{matrix} )}^{2} \qquad ( 13 )$$

where q and s are positive constants.
Using the Lagrange formula

$$x ( k + i ) = {A}_{IO}^{i} x ( k ) + {\sum }_{j = 0}^{i - 1} {A}_{IO}^{i - j - 1} ( {B}_{IO} u ( k + j ) + {M}_{IO} d ( k + j ) ) \\ and δ y ( k + i ) = {C}_{IO} {x}_{IO} ( k + i ) \\ we obtain \\ Y ( k ) = {A}_{L} {x}_{IO} ( k ) + {B}_{L} U ( k ) + {M}_{L} D ( k ) \\ Y ( k ) = [ \begin{matrix}δ y ( k + 1 ) \\ δ y ( k + 2 ) \\ ⋮ \\ δ y ( k + N - 1 ) \\ δ y ( k + N )\end{matrix} ] , \\ {A}_{L} = [ \begin{matrix}{C}_{IO} {A}_{IO} \\ {C}_{IO} {A}_{IO}^{2} \\ ⋮ \\ {C}_{IO} {A}_{IO}^{N - 1} \\ {C}_{IO} {A}_{IO}^{N}\end{matrix} ] \\ D ( k ) = [ \begin{matrix}d ( k ) \\ d ( k + 1 ) \\ ⋮ \\ d ( k + N - 1 ) \\ d ( k + N )\end{matrix} ] , \\ U ( k ) = [ \begin{matrix}δ u ( k ) \\ δ u ( k + 1 ) \\ ⋮ \\ δ u ( k + N - 1 ) \\ δ u ( k + N )\end{matrix} ] \\ {M}_{L} = [ \begin{matrix}{C}_{IO} {M}_{IO} & 0 & 0 & … & 0 & 0 & 0 \\ {C}_{IO} {A}_{IO} {M}_{IO} & {C}_{IO} {M}_{IO} & 0 & … & 0 & 0 & 0 \\ ⋮ & ⋮ & ⋮ & ⋮ & ⋮ & ⋮ & ⋮ \\ {C}_{IO} {A}_{IO}^{N - 2} {M}_{IO} & {C}_{IO} {A}_{IO}^{N - 3} {M}_{IO} & {C}_{IO} {A}_{IO}^{N - 4} {M}_{IO} & … & {C}_{IO} {M}_{IO} & 0 & 0 \\ {C}_{IO} {A}_{IO}^{N - 1} {M}_{IO} & {C}_{IO} {A}_{IO}^{N - 2} {M}_{IO} & {C}_{IO} {A}_{IO}^{N - 3} {M}_{IO} & … & {C}_{IO} {A}_{IO} {M}_{IO} & {C}_{IO} {M}_{IO} & 0\end{matrix} ] \\ Letting \\ {Y}^{o} ( k ) = [ \begin{matrix}{y}^{o} ( k + 1 ) \\ {y}^{o} ( k + 2 ) \\ ⋮ \\ {y}^{o} ( k + N - 1 ) \\ {y}^{o} ( k + N )\end{matrix} ] , \\ Q = [ \begin{matrix}q & 0 & … & 0 & 0 \\ 0 & q & … & 0 & 0 \\ … & … & ⋱ & … & … \\ 0 & 0 & … & q & 0 \\ 0 & 0 & … & 0 & s\end{matrix} ] , \\ R = [ \begin{matrix}r & 0 & … & 0 & 0 \\ 0 & r & … & 0 & 0 \\ … & … & ⋱ & … & … \\ 0 & 0 & … & r & 0 \\ 0 & 0 & … & 0 & r\end{matrix} ] , \\ \overline{Y} ( k ) = [ \begin{matrix}{G}_{b} ( k ) \\ {G}_{b} ( k ) \\ ⋮ \\ {G}_{b} ( k ) \\ {G}_{b} ( k )\end{matrix} ] \\ then \\ \begin{matrix}J ( {x}_{IO} ( k ) , u ) = \overline{J} ( {x}_{IO} ( k ) , u ) \\ = ( \begin{matrix}{Y}^{o} ( k ) - \\ {A}_{L} {x}_{IO} ( k ) - \\ {B}_{L} U ( k ) - \\ {M}_{L} D ( k )\end{matrix} ) Q ( \begin{matrix}{Y}^{o} ( k ) - \\ {A}_{L} {x}_{IO} ( k ) - \\ {B}_{L} U ( k ) - \\ {M}_{L} D ( k )\end{matrix} ) + {U}^{′} ( k ) RU ( k )\end{matrix} \qquad ( 14 )$$

The solution of the optimization problem has the following structure

δu⁰(k)=[10 . . . 0](B_{L}′QB_{L}+R)⁻¹B_{L}′Q(Y⁰(k)− Y̅(k)−A_{L}x_{IO}(k)−M_{L}D(k)) (15)

The injected insulin is then given by

δuₙₒₘ(t)=u_{b}(t)+δu⁰(t)

If the calculated insulin rate uₙₒₘ(t) is negative, a zero value will be applied to the system. In order to take into account of the effect of the saturation and to avoid wind-up problems the vector x_{IO} is obtained with the saturated value of the variable uₙₒₘ. The fulfillment of the state constraints, on the contrary, cannot be guaranteed; it is only possible to tune the parameter q so as to improve the regulation performance. The major advantages of this input-output MPC scheme are that an observer is not required (x_{IO} is made of past input and output values), and that it is easily implementable because real-time optimization is avoided. The possibility of considering time-varying basal glucose and basal insulin allows including a feedforward action computed to partially reject to meal and exercise disturbance.

It is possible to consider explicitly both input and state constraints by solving a constrained linear quadratic optimization problem with cost function (14).

MPC, in general, has several independent tuning parameters: control and prediction horizon, output and input weights, terminal penalty. However, possible choices are a prediction horizon equal to the control horizon between about 2 and about 4 hours, a terminal penalty s=q, and r=1. It should be appreciated that the prediction horizon and control horizon may be less than two hours or greater than four hours, as desired or required. The sampling time Ts can be chosen accordingly to the characteristic of the pump and the sensor. The sampling time can be changed without any problem during the commutation from a sampling time to another one. Remarkably the linearized model (12) is based on the mean value parameters of a particular population (for example different values for children and adults should be used) but it is not necessary to identify the particular model of each subject. Following these suggestions the only parameter to be tuned is the output weight q in a quite straightforward and intuitive way: a reduction of q makes the control action less aggressive, thus using less insulin. This implies an increase of both the minimum and the maximum value of the Glycemia.

In order to calibrate q a performance metric is needed. This is given for example by the so called Control Variability Grid Analysis (CVGA) [41] which takes into account both hypo- and hyper-glycemic extreme points during a prescribed observation period. The best q is the one that brings the patient closest to the lower left corner in the CVGA plot. The idea is to compute such optimal q from suitable patient's features. These features are either clinical parameters (see e.g. BW, TDI, CF, CR) or parameters obtained from insulin and glucose data collected during a screening visit (see e.g. AUC(G), AUC(G-Gₚᵣₑ), AUC(I), AUC(I-Iₚᵣₑ), ΔG, ΔI, T, SI). A rule is searched for that gives the optimal q as a function of the patient's features. The rule is obtained through the analysis of a virtual trial. The model describing a population of diabetic subjects, similar to the patient hand (e.g. adults or adolescent or children depending on the case) is used to extract a set of patients on which simulated closed-loop glucose control is applied. The patients of the trial, being randomly extracted, have different features and for each of them the optimal q parameter is obtained via a trial and error procedure. The output of the virtual trial is a set of patients with their individual features and the corresponding optimal q parameters. Statistical regression is used to obtain the relationship that links patient's features to the best q parameter. The relationship can take the form of a log-log linear regression linking the logarithm of patient's features to the log q. In order to avoid overparametrization and select only a subset of relevant parameters, stepwise regression is used.

### REFERENCES

It should be appreciated that various aspects of embodiments of the present method, system, devices and computer program product may be implemented with the following methods, systems, devices and computer program products disclosed in the following U.S. Patent Applications, U.S. Patents, and PCT International Patent Applications that are hereby incorporated by reference herein and co-owned with the assignee:

PCT/US2008/067725, entitled “Method, System and Computer Simulation Environment for Testing of Monitoring and Control Strategies in Diabetes,” filed Jun. 20, 2008;

PCT/US2007/085588 not yet published filed Nov. 27, 2007, entitled “Method, System, and Computer Program Product for the Detection of Physical Activity by Changes in Heart Rate, Assessment of Fast Changing Metabolic States, and Applications of Closed and Open Control Loop in Diabetes;”

U.S. Ser. No. 11/943,226, filed Nov. 20, 2007, entitled “Systems, Methods and Computer Program Codes for Recognition of Patterns of Hyperglycemia and Hypoglycemia, Increased Glucose Variability, and Ineffective Self-Monitoring in Diabetes;”

PCT International Application Serial No. PCT/US2005/013792, filed Apr. 21, 2005, entitled “Method, System, and Computer Program Product for Evaluation of the Accuracy of Blood Glucose Monitoring Sensors/Devices;”

U.S. patent application Ser. No. 11/578,831, filed Oct. 18, 2006 entitled “Method, System and Computer Program Product for Evaluating the Accuracy of Blood Glucose Monitoring Sensors/Devices;”

PCT International Application Serial No. PCT/US01/09884, filed Mar. 29, 2001, entitled “Method, System, and Computer Program Product for Evaluation of Glycemic Control in Diabetes Self-Monitoring Data;”

U.S. Pat. No. 7,025,425 B2 issued Apr. 11, 2006, entitled “Method, System, and Computer Program Product for the Evaluation of Glycemic Control in Diabetes from Self-Monitoring Data;”

U.S. patent application Ser. No. 11/305,946 filed Dec. 19, 2005 entitled “Method, System, and Computer Program Product for the Evaluation of Glycemic Control in Diabetes from Self-Monitoring Data” (Publication No. 2006/0094947);

PCT International Application Serial No. PCT/US2003/025053, filed Aug. 8, 2003, entitled “Method, System, and Computer Program Product for the Processing of Self-Monitoring Blood Glucose (SMBG) Data to Enhance Diabetic Self-Management;”

U.S. patent application Ser. No. 10/524,094 filed Feb. 9, 2005 entitled “Managing and Processing Self-Monitoring Blood Glucose” (Publication No. 2005/214892); PCT International Application Serial No PCT/US2006/033724, filed Aug. 29, 2006, entitled “Method for Improvising Accuracy of Continuous Glucose Sensors and a Continuous Glucose Sensor Using the Same;”

PCT International Application No. PCT/US2007/000370, filed Jan. 5, 2007, entitled “Method, System and Computer Program Product for Evaluation of Blood Glucose Variability in Diabetes from Self-Monitoring Data;”

U.S. patent application Ser. No. 11/925,689, filed Oct. 26, 2007, entitled “For Method, System and Computer Program Product for Real-Time Detection of Sensitivity Decline in Analyte Sensors;”

PCT International Application No. PCT/US00/22886, filed Aug. 21, 2000, entitled “Method and Apparatus for Predicting the Risk of Hypoglycemia;”

U.S. Pat. No. 6,923,763 B1, issued Aug. 2, 2005, entitled “Method and Apparatus for Predicting the Risk of Hypoglycemia;” and

PCT International Patent Application No. PCT/US2007/082744, filed Oct. 26, 2007, entitled “For Method, System and Computer Program Product for Real-Time Detection of Sensitivity Decline in Analyte Sensors.”

### REFERENCES CITED

The following patents, applications and publications as listed below and throughout this document are hereby incorporated by reference in their entirety herein.

The devices, systems, compositions and methods of various embodiments of the invention disclosed herein may utilize aspects disclosed in the following references, applications, publications and patents and which are hereby incorporated by reference herein in their entirety:

### U.S. Patent Documents

U.S. Pat. No. 7,299,082, filed October 2007, to Feldman et al.

U.S. Pat. No. 6,558,351, filed May 2003, to Steil et al

U.S. Pat. No. 6,544,212, filed April 2003, to Galley et al.

U.S. Pat. No. 5,660,163, filed August 1997, to Schulman et al

### Foreign Patent Documents

PCT Application No. PCT/US2006/033724 (Publication No. WO 2007/027691) “Improving the Accuracy of Continuous Glucose Sensors” published Mar. 8, 2007.

PCT Application No. PCT/US2007/082744 (Publication No. WO 2008/052199) “Method, System and Computer Program Product for Real-Time Detection of Sensitivity Decline in Analyte Sensors” published May 2, 2008.

### OTHER PUBLICATIONS

1. Pfeiffer E F, Thum C h, and Clemens A H, “The artificial beta cell—A continuous control of blood sugar by external regulation of insulin infusion (glucose controlled insulin infusion system)”, Horm Metab Res 487: 339-342, 1974.
2. Clemens A H, “Feedback control dynamics for glucose controlled insulin infusion system”, MedProg Technol 6: 91-98, 1979.
3. Albisser A M, Leibel B S, Ewart T G, Davidovac Z, Botz C K, and Zingg W, “An artificial endocrine pancreas”, Diabetes, 23: 389-396, 1974.
4. Marliss E B, Murray F T, Stokes E F, Zinman B, Nakhooda A F, Denoga A, Leibel B S, and Albisser A M, “Normalization of glycemia in diabetics during meals with insulin and glucagon delivery by the artificial pancreas”, Diabetes 26: 663-672, 1977.
5. Kraegen E W, Campbell L V, Chia Y O, Meler H, and Lazarus L, “Control of blood glucose in diabetics using an artificial pancreas”, Aust N Z J Med 7: 280-286, 1977.
6. Fischer U, Jutzi E, Freyse E-J, and Salzsieder E, “Derivation and experimental proof of a new algorithm for the Artificial®-cell based on the individual analysis of the physiological insulin-glucose relationship”, Endokrinologie 71:65-75, 1978.
7. Broekhuyse H M, Nelson J D, Zinman B, and Albisser A M, “Comparison of algorithms for the closed-loop control of blood glucose using the artificial beta cell”, IEEE Trans Biomed Eng BME-28: 678-687, 1981.
8. Salzsieder E, Albrecht G, Fischer U, and Freyse E-J, “Kinetic modeling of the glucoregulatory system to improve insulin therapy”, IEEE Trans Biomed Eng 32: 846-855, 1985.
9. Fischer U, Schenk W, Salzsieder E, Albrecht G, Abel P, and Freyse E-J, “Does physiological blood glucose control require an adaptive strategy?”, IEEE Trans Biomed Eng BME-34:575-582, 1987.
10. Sorensen J T, “A Physiologic Model of Glucose Metabolism in Man and its Use to Design and Assess Improved Insulin Therapies for Diabetes”, Ph.D. dissertation, Department of Chemical Engineering, MIT, 1985.
11. Swan G, “An optimal control model of diabetes mellitus”, Bull Math Bio 44: 793-808, 1982.
12. Fisher M E and Teo K L, “Optimal insulin infusion resulting from a mathematical model of blood glucose dynamics”, IEEE Trans Biomed Eng 36: 479-486, 1989.
13. Ollerton R L, “Application of optimal control theory to diabetes mellitus”, Int J Control 50: 2503-2522, 1989.
14. Fisher M E, “A semi closed-loop algorithm for the control of blood glucose levels in diabetics”, IEEE Trans Biomed Eng 38: 57-61, 1991.
15. Kienitz K H and Yoneyama T, “A robust controller for insulin pumps based on H-infinity theory”, IEEE Trans Biomed Eng 40: 1133-1137, 1993.
16. Sorensen J. T.,: A physiologic model of glucose metabolism in man and its use to design and assess improved insulin therapies for diabetes”, in Department of Chemical Engineering. 1985, MIT.
17. Parker R S, Doyle III F J, and Peppas N A, “A model-based algorithm for blood glucose control in type I diabetic patients”, IEEE Trans Biomed Eng 46(2): 148-157, 1999.
18. El-Khatib F H, Jiang J, Damiano E R, “Adaptive closed-loop control provides blood-glucose regulation using dual subcutaneous insulin and glucagon infusion in diabetic swine”, Diabetes Sci Tech, 1:181-192, 2007.
19. Hovorka R., Canonico V., Chassin L. J., Haueter U., Massi-Benedetti M., Federici M. O., Pieber T. R., Schaller H. C., Schaupp L., Vering T., “Nonlinear model predictive control of glucose concentration in subjects with type 1 diabetes”, Phsyiological Measurement, 25 (4) 905-920, 2004.
20. Leblanc H, Chauvet D, Lombrail P, Robert J J, “Glycemic control with closed-loop intraperitoneal insulin in type I diabetes”. Diabetes Care, 9: 124-128, 1986.
21. J L Selam, P Micossi, F L Dunn, and D M Nathan, “Clinical trial of programmable implantable insulin pump for type I diabetes”, Diabetes Care 15: 877-885. 1992.
22. E Renard, “Implantable closed-loop glucose-sensing and insulin delivery: the future for insulin pump therapy”, Current Opinion in Pharmacology, 2(6), 708-716, 2002.
23. R. Hovorka, “Continuous glucose monitoring and closed-loop systems”, Diabetic Medicine 23 (1), 1-12. 2006.
24. Magni L., G. De Nicolao, L. Magnani, R. Scattolini: “A Stabilizing Model-Based Predictive Controller for Nonlinear Systems”, Automatica, 37, 11351-1362, 2001.
25. De Nicolao G., L. Magni, R. Scattolini: “Stabilizing Receding-Horizon Control of Nonlinear Time-Varying Systems”, IEEE-AC, 1030-1036, 1998.
26. Magni L., R. Sepulchre “Stability margin of Nonlinear Receding Horizon Control via Inverse Optimality”, Syst. & Contr. Letters, 32, 241-245, 1997.
27. Steil G M, Rebrin K, Darwin C, Hariri F, Saad M F, “Feasibility of automating insulin delivery for the treatment of type 1 diabetes”, Diabetes. December; 55(12):3344-50, 2006.
28. Mayne, D. Q., J. B. Rawlings, C. V. Rao and P. O. M. Scokaert, “Constrained model predictive control: Stability and optimality”, Automatica, 2000, 36, 789-814.
29. Garcia C. E., D. M. Prett, and M. Morari, “Model predictive control theory and practice—A survey,” Automatica, vol. 25, no. 3, pp. 335-348, 1989.
30. Dua, P., F. J. Doyle III and E. N. Pistikopoulos, “Model-based blood glucose control for type 1 diabetes via parametric programming”, IEEE Transactions on Biomedical Engineering 2006, 53, 1478-1491.
31. Dalla Man, C., R. A. Rizza and C. Cobelli, “Meal simulation model of the glucose-insulin system”, IEEE Transactions on Biomedical Engineering, 2007, 54, 1740-1749.
32. Dalla Man, C., M. Camilleri and C. Cobelli, “A system model of oral glucose absorption: validation on gold standard data”, IEEE Transactions on Biomedical Engineering 2006, 53, 2472-2478.
33. Vicini, P., A. Caumo and C. Cobelli, “The hot IVGTT two-compartment minimal model: Indexes of glucose effectiveness and insulin sensitivity”, Am J. Physiol. 1997, 273, E1024-32.
34. Breda, E., M. K. Cavaghan, G. Toffolo, K. S. Polonsky and C. Cobelli, “Oral glucose tolerance test minimal model indexes of beta-cell function and insulin sensitivity”, Diabetes 2001, 50, 150-158.
35. Dalla Man, C., G. Toffolo, R. Basu, R. A Rizza and C. Cobelli, “A model of glucose production during a meal,” In: Proceedings of IEEE-EMBS Conference, New York. 2006.
36. Verdonk, C. A., R. A Rizza and J. E. Gerich, “Effects of plasma glucose concentration on glucose utilization and glucose clearance in normal man”, Diabetes 1981, 30, 535-537.
37. Trajanoski Z. and Wach P., “Neural predictive controller for insulin delivery using the subcutaneous route”, IEEE Trans Biomed Eng, 1998. 45(9): p. 1122-34.
38. Kan S., Onodera H., Furutani E., et al., “Novel control system for blood glucose using a model predictive method”, Asaio J, 2000. 46(6): p. 657-62.
39. Bequette, B. W., “Nonlinear control of chemical processes: a review”, Ind. Eng. Chem. Res., 1991. 30(7): p. 1391-1413.
40. Kuure-Kinsey M. R., Cutright R., and Bequette B. W., “Computationally Efficient Neural Predictive Control based on a Feedforward Architecture”, in 2006 American Control Conference. 2006. Minneapolis, Minn.
41. Magni L., Davide M. Raimondo, Chiara Dalla Man, Marc Breton, Stephen Patek, Giuseppe De Nicolao, Claudio Cobelli, Boris Kovatchev: “Evaluating the efficacy of closed-loop glucose regulation via Control-Variability Grid Analysis (CVGA)”, Journal of Diabetes Science and Technology, 2, 630-635, 2008.
42. Magni L., D. M. Raimondo, L. Bossi, C. Dalla Man, G. De Nicolao, B. Kovatchev and C. Cobelli: “Model Predictive Control of Type 1 Diabetes: An in Silico Trial”, Journal of Diabetes Science and Technology, 1, 804-812, 2007.

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes which come within the meaning and range of equivalency of the claims are therefore intended to be embraced herein.

It should be appreciated that the system and methods described herein, though focused primarily on uses that utilize continuous glucose monitoring, may also be utilized with an SMBG implementation or a combination of SMBG and continuous glucose monitoring.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A system for providing optimal insulin injections to a subject to be used with a continuous glucose monitor (CGM) and an insulin delivery unit, said system comprising:
a controller, wherein said controller comprises:
a discrete-time, linear model predictive control law,
means for sending information to said insulin delivery unit, and
means for receiving information from said CGM.

2. The system of claim 1, wherein said control law is derived from a discrete-time model of glucose insulin dynamics and an aggressiveness parameter.

3. The system of claim 2, wherein said control law is derived from said discrete-time model of glucose insulin dynamics by linearizing a model about an equilibrium point that is associated with the average basal values of a population model.

4. The system of claim 3, wherein said control law may be expressed as
u=κ^{MPC}(x).

5. The system of claim 2, wherein said aggressiveness parameter is determined from data that is individualized to said subject.

6. The system of claim 5, wherein said aggressiveness parameter is determined according to suitable features of the subject, wherein said features comprise one or more of the following input parameters:
clinical parameters including but not limited to body weight, average total daily utilization insulin, and carbohydrate ratio and
parameters obtained from insulin and glucose data collected during a screening visit.

7. The system of claim 6, wherein said aggressiveness parameter is given by the equation
$$q = exp ( {k}_{0} + {\sum }_{i = 1}^{n} {k}_{i} \cdot ln ( {θ}_{i} ) )$$
and wherein kᵢ i=1, . . . n, are regression coefficients and θᵢ i=1, . . . n, are said input parameters.

8. The system of claim 7, wherein one or more of said regression coefficients are selected according to whether the subject is a member of one or more of a set of predefined classes.

9. The system of claim 8, wherein said set of predefined classes includes one or more of the following: child, adolescent, and adult.

10. The system of claim 2, wherein said aggressiveness parameter is determined off-line.

11. The system of claim 2, wherein said aggressiveness parameter represents how aggressively the controller should adjust its insulin output to achieve a desired glucose level in a subject.

12. The system of claim 2, wherein said discrete-time model of glucose insulin dynamics describes deviations from the subject's fasting glucose concentration and basal insulin rate.

13. The system of claim 12, wherein said discrete-time model of glucose insulin dynamics is represented by the following state space equations:
δx(k+1)=A_{D}δx(k)+B_{Du}δu(k)+B_{Dd}d(k)
δy(k)=C_{D}δx(k)
where δx(k)=x(kTₛ)− x̅, δu(k)=u(kTₛ)−ū and δy(k)=y(kTₛ)− y̅.

14. The system of claim 2, wherein, for a given stage corresponding to a discrete time period, using said control law, a first insulin rate is determined by solving a finite horizon optimal control problem so that a cost function is minimized.

15. The system of claim 14, wherein, for a given stage corresponding to a discrete time period, said first insulin rate is determined by considering a set of parameters, said set of parameters comprising one or more of the following:
a state vector,
target glucose concentration, and
future glucose disturbances.

16. The system of claim 15, wherein said state vector is expressed as:
$${x}_{IO} ( k ) = [ \begin{matrix}δ y ( k ) \\ ⋮ \\ δ y ( k - n + 1 ) \\ δ u ( k - 1 ) \\ ⋮ \\ δ u ( k - n + 1 ) \\ d ( k - 1 ) \\ ⋮ \\ d ( k - n + 1 )\end{matrix} ] .$$

17. The system of claim 15, wherein said vector of target glucose concentrations is expressed as:
$${Y}^{o} ( k ) = [ \begin{matrix}{y}^{o} ( k + 1 ) \\ {y}^{o} ( k + 2 ) \\ ⋮ \\ {y}^{o} ( k + N - 1 ) \\ {y}^{o} ( k + N )\end{matrix} ] .$$

18. The system of claim 15, wherein said future glucose disturbances are expressed as:
$$D ( k ) = [ \begin{matrix}d ( k ) \\ d ( k + 1 ) \\ ⋮ \\ d ( k + N - 1 ) \\ d ( k + N )\end{matrix} ] .$$

19. The system of claim 15, wherein said future glucose disturbances represent meal announcements.

20. The system of claim 15, wherein said cost function is expressed as:
$$J ( {x}_{IO} ( k ) , δ u ( \cdot ) ) = {\sum }_{i = 0}^{N - 1} ( q \begin{matrix}{( \begin{matrix}{y}^{0} ( k + i ) - \\ y ( k + i )\end{matrix} )}^{2} + \\ {r ( δ u ( k + i ) )}^{2}\end{matrix} ) + {s ( \begin{matrix}{y}^{0} ( k + N ) - \\ y ( k + N )\end{matrix} )}^{2} .$$

21. The system of claim 15, wherein said first insulin rate is determined by considering a set of additional operational parameters, said set of additional operational parameters comprising one or more of the following:
upper limit on the allowable glucose level in the subject,
lower limit on the allowable glucose level in the subject,
prediction horizon for achieving target glucose level, and
control horizon for future optimal insulin injections.

22. The system of claim 21, wherein said additional operational parameters have associated weight factors which indicate their relative importance.

23. The system of claim 21, wherein said prediction horizon is between about two and about four hours.

24. The system of claim 21, wherein said control horizon is between about two and about four hours.

25. The system of claim 15, wherein a second insulin rate is determined by applying discretization and safety filters to said first insulin rate.

26. The system of claim 25, wherein said safety filters include one or more of the following:
ensure that the rate of insulin applied does not exceed a certain limit within a certain time period,
ensure that the rate of insulin applied does not exceed a certain limit within a certain time period after a meal, and
ensure that basal rate does not exceed a certain percentage of the subject specified basal rate per hour.

27. The system of claim 25, wherein said safety filters include a safety filter to ensure that no more than about 3 units of bolus insulin are applied within a one hour period.

28. The system of claim 25, wherein said safety filters include a safety filter to ensure that no more than about 10 units of bolus insulin per hour (not counting basal insulin) are applied within about 2 hours of a meal.

29. The system of claim 25, wherein said safety filters include a safety filter to ensure that the basal rate does not exceed about 150% of the subject's specified basal rate per hour.

30. The system of claim 25, wherein the controller sends information to the insulin delivery unit based upon the second insulin rate, said information indicating a current optimal insulin injection.

31. The system of claim 1, wherein said control law is derived from a continuous-time model of glucose insulin dynamics and an aggressiveness parameter.

32. The system of claim 1, wherein the controller receives information from said CGM at regular time intervals.

33. The system of claim 32, wherein said time intervals are approximately one minute apart.

34. The system of claim 32, wherein the duration of said time intervals may be varied.

35. The system of claim 1, wherein the controller sends information to said insulin delivery unit at regular time intervals.

36. The system of claim 35, wherein said time intervals are approximately fifteen minutes apart.

37. The system of claim 35, wherein the duration of said regular time intervals may be varied.

38. The system of claim 1, wherein the controller receives information from the CGM through a wireless connection.

39. The system of claim 1, wherein the controller receives information from the CGM through a wired connection.

40. The system of claim 1, wherein the controller communicates with the insulin delivery unit through a wireless connection.

41. The system of claim 1, wherein the controller communicates with the insulin delivery unit through a wired connection.

42. The system of claim 1, wherein the system is fully within the body of the subject.

43. The system of claim 1, wherein the system is partially within the body of the subject.

44. The system of claim 1, wherein the controller is within or attached to said CGM.

45. The system of claim 1, wherein the controller is within or attached to said insulin delivery unit.

46. The system of claim 1, wherein said insulin delivery unit delivers insulin to the subject upon receiving a command from the controller.

47. The system of claim 1, wherein said insulin delivery unit is comprised of an insulin pump.

48. The system of claim 47, wherein said insulin pump is the Omnipod from Insulet corporation.

49. The system of claim 47, wherein said insulin pump is the Deltec Cozmo from Smiths Medical.

50. The system of claim 1, wherein said insulin delivery unit comprises an insulin reservoir.

51. The system of claim 1, wherein said insulin delivery unit comprises a cannula for subcutaneous insertion.

52. The system of claim 1, wherein said CGS is the Navigator from Abbott Diabetes Care.

53. The system of claim 1, wherein said CGS is the Dexcom from Dexcom, Inc.

54. The system of claim 1, wherein said CGS is the Guardian/Paradigm from Medtronic.

55. The system of claim 1, wherein said subject is a human being.

56. A system for providing optimal insulin injections to a subject, said system comprising:
a continuous glucose monitor (CGM),
an insulin delivery unit, and
a controller, wherein said controller comprises:
a discrete-time, linear model predictive control law,
means for sending information to said insulin delivery unit, and
means for receiving information from said CGM.

57. A system for providing optimal insulin injections to a subject to be used with a continuous glucose monitor (CGM), said system comprising:
an insulin delivery unit, and
a controller, wherein said controller comprises:
a discrete-time, linear model predictive control law,
means for sending information to said insulin delivery unit, and
means for receiving information from said CGM.

58. A system for providing optimal insulin injections to a subject to be used with an insulin delivery unit, said system comprising:
a continuous glucose monitor (CGM), and
a controller, wherein said controller comprises:
a discrete-time, linear model predictive control law,
means for sending information to said insulin delivery unit, and
means for receiving information from said CGM.

59. A computer method for providing optimal insulin injections to a subject to be used with a continuous glucose monitor (CGM) and an insulin delivery unit, said method comprising:
providing a discrete time linear model predictive control law,
sending information to an insulin delivery unit, and
receiving information from the CGM.

60. The method of claim 59, wherein said control law is derived from a discrete-time model of glucose insulin dynamics and an aggressiveness parameter.

61. The method of claim 60, wherein said control law is derived from said discrete-time model of glucose insulin dynamics by linearizing a model about an equilibrium point that is associated with the average basal values of a population model.

62. The method of claim 61, wherein said control law may be expressed as
u=κ^{MPC}(X).

63. The method of claim 60, wherein said aggressiveness parameter is determined from data that is individualized to said subject.

64. The method of claim 63, wherein said aggressiveness parameter is determined according to suitable features of the subject, wherein said features comprise one or more of the following input parameters:
clinical parameters including but not limited to body weight, average total daily utilization insulin, and carbohydrate ratio, and parameters obtained from insulin and glucose data collected during a screening visit.

65. The system of claim 64, wherein said aggressiveness parameter is given by the equation
$$q = exp ( {k}_{0} + {\sum }_{i = 1}^{n} {k}_{i} \cdot ln ( {θ}_{i} ) )$$
and wherein kᵢ i=1, . . . n, are regression coefficients and θᵢ i=1, . . . n, are said input parameters.

66. The method of claim 65, wherein one or more of said regression coefficients are selected according to whether the subject is a member of one or more of a set of predefined classes.

67. The method of claim 66, wherein said set of predefined classes includes one or more of the following: child, adolescent, and adult.

68. The method of claim 60, wherein said aggressiveness parameter is determined off-line.

69. The method of claim 60, wherein said aggressiveness parameter represents how aggressively the controller should adjust its insulin output to achieve a desired glucose level in a subject.

70. The method of claim 60, wherein said discrete-time model of glucose insulin dynamics describes deviations from the subject's fasting glucose concentration and basal insulin rate.

71. The method of claim 70, wherein said discrete-time model of glucose insulin dynamics is represented by the following state space equations:
δx(k+1)=A_{D}δx(k)+B_{Du}δu(k)+B_{Dd}d(k)
δy(k)=C_{D}δx(k)
where δx(k)=x(kTₛ)− x̅, δu(k)=u(kTₛ)−ū and δy(k)=y(kTₛ)− y̅.

72. The method of claim 60, wherein said method further comprising determining a first insulin rate by solving a finite horizon optimal control problem so that a cost function is minimized.

73. The method of claim 72, further comprising determining said first insulin rate by considering a set of parameters, said set of parameters comprising one or more of the following:
a state vector,
target glucose concentration, and
future glucose disturbances.

74. The method of claim 73, wherein said state vector is expressed as:
$${x}_{IO} ( k ) = [ \begin{matrix}δ y ( k ) \\ ⋮ \\ δ y ( k - n + 1 ) \\ δ u ( k - 1 ) \\ ⋮ \\ δ u ( k - n + 1 ) \\ d ( k - 1 ) \\ ⋮ \\ d ( k - n + 1 )\end{matrix} ] .$$

75. The method of claim 73, wherein said vector of target glucose concentrations is expressed as:
$${Y}^{o} ( k ) = [ \begin{matrix}{y}^{o} ( k + 1 ) \\ {y}^{o} ( k + 2 ) \\ ⋮ \\ {y}^{o} ( k + N - 1 ) \\ {y}^{o} ( k + N )\end{matrix} ] .$$

76. The method of claim 73, wherein said future glucose disturbances are expressed as:
$$D ( k ) = [ \begin{matrix}d ( k ) \\ d ( k + 1 ) \\ ⋮ \\ d ( k + N - 1 ) \\ d ( k + N )\end{matrix} ] .$$

77. The method of claim 73, wherein said future glucose disturbances represent meal announcements.

78. The method of claim 73, wherein said cost function is expressed as:
$$J ( {x}_{IO} ( k ) , δ u ( \cdot ) ) = {\sum }_{i = 0}^{N - 1} ( q \begin{matrix}{( \begin{matrix}{y}^{0} ( k + i ) - \\ y ( k + i )\end{matrix} )}^{2} + \\ {r ( δ u ( k + i ) )}^{2}\end{matrix} ) + {s ( \begin{matrix}{y}^{0} ( k + N ) - \\ y ( k + N )\end{matrix} )}^{2} .$$

79. The method of claim 73, further comprising determining said first insulin rate by considering a set of additional operational parameters, said set of additional operational parameters comprising one or more of the following:
upper limit on the allowable glucose level in the subject,
lower limit on the allowable glucose level in the subject,
prediction horizon for achieving target glucose level, and
control horizon for future optimal insulin injections.

80. The method of claim 79, wherein said additional operational parameters have associated weight factors which indicate their relative importance.

81. The method of claim 79, wherein said prediction horizon is between about two and about four hours.

82. The method of claim 79, wherein said control horizon is between about two and about four hours.

83. The method of claim 73, further comprising determining a second insulin rate by applying discretization and safety filters to said first insulin rate.

84. The method of claim 83, wherein said safety filters include one or more of the following:
ensuring that the rate of insulin applied does not exceed a certain limit within a certain time period,
ensuring that the rate of insulin applied does not exceed a certain limit within a certain time period after a meal, and
ensuring that basal rate does not exceed a certain percentage of the subject specified basal rate per hour.

85. The method of claim 83, wherein said safety filters include a safety filter ensuring that no more than about 3 units of bolus insulin are applied within a one hour period.

86. The method of claim 83, wherein said safety filters include a safety filter ensuring that no more than about 10 units of bolus insulin per hour (not counting basal insulin) are applied within about 2 hours of a meal.

87. The method of claim 83, wherein said safety filters include a safety filter ensuring that the basal rate does not exceed about 150% of the subject's specified basal rate per hour.

88. The method of claim 83, further comprising sending information to the insulin delivery unit based upon the second insulin rate, said information indicating a current optimal insulin injection.

89. The method of claim 59, wherein said control law is derived from a continuous-time model of glucose insulin dynamics and an aggressiveness parameter.

90. The method of claim 59, further comprising sending information from said CGM at regular time intervals.

91. The method of claim 90, wherein said time intervals are approximately one minute apart.

92. The method of claim 90, wherein the duration of said time intervals may be varied.

93. The method of claim 59, further comprising sending information to said insulin delivery unit at regular time intervals.

94. The method of claim 93, wherein said time intervals are approximately fifteen minutes apart.

95. The method of claim 93, wherein the duration of said regular time intervals may be varied.

96. The method of claim 59, further comprising receiving information from the CGM through a wireless connection.

97. The method of claim 59, further comprising receiving information from the CGM through a wired connection.

98. The method of claim 59, further comprising communicating with the insulin delivery unit through a wireless connection.

99. The method of claim 59, further comprising communicating with the insulin delivery unit through a wired connection.

100. The method of claim 59, wherein the method steps are performed within the body of the subject.

101. The method of claim 59, wherein the method steps are partially performed within the body of the subject.

102. The method of claim 59, wherein said controlling occurs within said CGM or in external communication with said CGM.

103. The method of claim 59, wherein said controlling occurs within said insulin delivery unit or in external communication with said insulin delivery unit.

104. The method of claim 59, wherein said insulin delivery unit delivers insulin to said subject upon receiving a command.

105. The method of claim 59, wherein said insulin delivery unit comprises an insulin pump.

106. The method of claim 105, wherein said insulin pump is the Omnipod from Insulet corporation.

107. The method of claim 105, wherein said insulin pump is the Deltec Cozmo from Smiths Medical.

108. The method of claim 59, wherein said insulin delivery unit comprises an insulin reservoir.

109. The method of claim 59, wherein said insulin delivery unit comprises a cannula for subcutaneous insertion.

110. The method of claim 59, wherein said CGS is the Navigator from Abbott Diabetes Care.

111. The method of claim 59, wherein said CGS is the Dexcom from Dexcom, Inc.

112. The method of claim 59, wherein said CGS is the Guardian/Paradigm from Medtronic.

113. The method of claim 59, wherein said subject is a human being.

114. A computer method for providing optimal insulin injections to a subject, said method comprising:
performing continuous glucose monitor monitoring,
performing insulin delivery,
providing a discrete time linear model predictive control law,
sending information to an insulin delivery unit, and
receiving information from a CGM.

115. A computer system for providing optimal insulin injections to a subject to be used with a continuous glucose monitor (CGM) said system comprising:
performing insulin delivery,
providing a discrete time linear model predictive control law,
sending information to an insulin delivery unit, and
receiving information from a CGM.

116. A method for providing optimal insulin injections to a subject to be used with an insulin delivery unit, said system comprising:
performing continuous glucose monitor monitoring,
providing a discrete time linear model predictive control law,
sending information to an insulin delivery unit, and
receiving information from a CGM.

117. A computer readable medium for use with a processor, to be used with a continuous glucose monitor (CGM) and an insulin delivery unit, having computer executable instructions for performing a method for computing an optimal adapting insulin injection, wherein said method comprises:
providing a discrete time linear model predictive control law
sending information to an insulin delivery unit, and
receiving information from the CGM.

118. The computer readable medium of claim 117, wherein said control law is derived from a discrete-time model of glucose insulin dynamics and an aggressiveness parameter.

119. The computer readable medium of claim 118, wherein said control law is derived from said discrete-time model of glucose insulin dynamics by linearizing a model about an equilibrium point that is associated with the average basal values of a population model.

120. The computer readable medium of claim 118, wherein said computer readable medium further contains instructions for determining a first insulin rate by solving a finite horizon optimal control problem so that a cost function is minimized.

121. The computer readable medium of claim 120, wherein said computer readable medium further contains instructions for determining said first insulin rate by considering a set of parameters, said set of parameters comprising one or more of the following:
a state vector,
target glucose concentration, and
future glucose disturbances.
